(19) United States
(12) Patent Application Publication
(10) Pub. No.: US 2011/0151034 A1
(43) Pub. Date: Jun. 23, 2011

(54) ANTIBACTERIAL AGENT AND DISINFECTING METHOD

(75) Inventors: Hirotsugu Kido, Kanagawa (JP); Shigeo Ii, Kanagawa (JP)

(73) Assignees: Mitsubishi Chemical Corporation, Tokyo (JP); Mitsubishi-Kagaku Foods Corporation, Tokyo (JP)

(21) Appl. No.: 13/057,658

(22) PCT Filed: Aug. 3, 2009
(86) PCT No.: PCT/JP2009/063764

§ 371 Date: Feb. 4, 2011

(30) Foreign Application Priority Data
Aug. 4, 2008 (JP) 2008-201147

(51) Int. Cl. A01N 65/22; A23L 3/349; C07C 39/17; A01P 1/00

(52) U.S. Cl. 424/745; 424/746; 426/335; 568/719

(57) ABSTRACT

To provide an antibacterial agent effective against gram-positive bacteria, particularly an antibacterial agent that is safe even when added to foods and the like, and a disinfecting method.

An antibacterial agent comprising as an active ingredient either one or both of compounds represented by the following formula (1) and (2) contained in a labiatae plant extract:

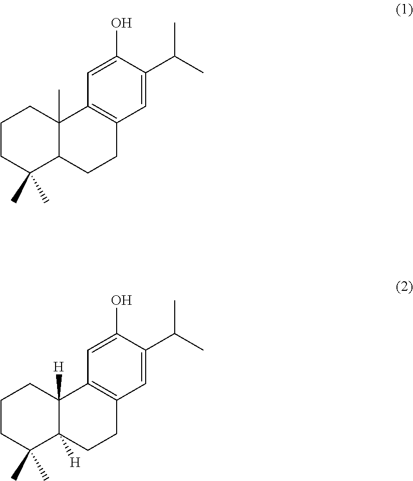

This antibacterial agent has antibacterial activity against gram-positive bacteria belonging to the genus Staphylococcus, the genus Lactobacillus, the genus Listeria, the genus Alicyclobacillus or the genus Bacillus.

### TECHNICAL FIELD

The present invention relates to an antibacterial agent and a disinfecting method. More particularly, it relates to an antibacterial agent comprising as an active ingredient a specific compound contained in a labiatae plant extract, and a disinfecting method employing it.

### BACKGROUND ART

It is known from recent studies that listeriosis in humans caused by the genus Listeria particularly Listeria monocytogenes relates to ingestion of various foods particularly soft cheese, pâté, ham and other prepackaged butcher meat and poultry products (e.g. studies by PHLS (Public Health Laboratory Service) on 16 food chain restaurants (Non-Patent Document 1)). Further, the main contaminant source of the genus Listeria is generally considered to be food manufacturers.

Listeria monocytogenes which is a food-borne pathogenic bacterium, causes about 70 deaths annually in Japan, which is higher than double the deaths by other food-borne pathogenic bacteria. Further, in the United States, as of 2000, the number of deaths by Listeria food poisoning amounted to about 500, and the damage by Listeria monocytonegenes is comparable to those by Salmonella and O157 which is an enterohemorrhagic strain of the bacterium Escherichia coli.

On the other hand, hop acid and an acid similar to hop acid have been recognized as a bacteria inhibitor (Patent Document 1).

### PRIOR ART DOCUMENT

### Patent Document

Patent Document 1: JP-A-8-502887

### Non-Patent Document

Non-Patent Document 1: Listeria Monocytogenes in Prepacked Ready-To-Eat Sliced Meat, S. Velani and R. J. Gilbert, PHLS Microbiology Digest, vol. 7 (1977)

### DISCLOSURE OF THE INVENTION

### Problems to be Solved by the Invention

However, antibacterial agents (particularly antibacterial agents derived from natural products) have been known to differ in the antibacterial activity depending on the type of the microorganism, and although conventional antibacterial agents have antibacterial activity against Salmonella and the enterohemorrhagic strain of Escherichia coli, not so many antibacterial agents effective against gram-positive bacteria such as bacteria belonging to the genus Listeria, particularly antibacterial agents derived from natural products that are safe even when added to foods and the like, have been known.

Further, hop acid (particularly βacid-containing hop extract) which is an antibacterial agent derived from natural products has insufficient antibacterial activity against the genus Listeria. Further, hop acid which itself has a specific odor, is inappropriate as an antibacterial agent to be added to foods.

That is, the object of the present invention is to provide an antibacterial agent having antibacterial activity against gram-positive bacteria, substantially free from odor, and derived from natural products so that it is safe even when added to foods and the like.

### Means to Solve the Problems

The present inventors have conducted extensive studies to solve the above problems and as a result, found that a certain compound contained in a labiatae plant extract, for example, a rosemary extract, has excellent antibacterial activity against gram-positive bacteria such as bacteria belonging to the genus Listeria. The present invention has been accomplished on the basis of this discovery.

That is, the present invention provides the following [1] to [13].

[1] An antibacterial agent comprising as an active ingredient either one or both of compounds represented by the following formulae (1) and (2) contained in a labiatae plant extract:

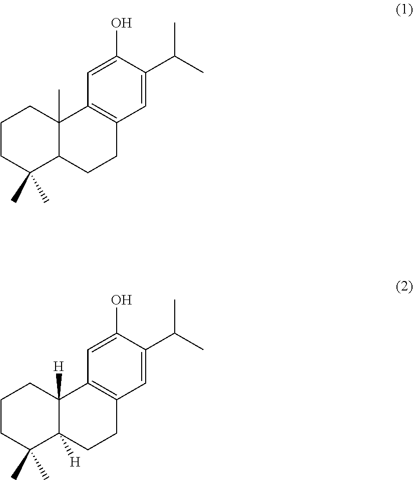

[2] The antibacterial agent according to the above [1], wherein the labiatae plant extract is at least one extract selected from the group consisting of a rosemary extract, a sage extract, a thyme extract and an oregano extract.
[3] An antibacterial agent comprising as an active ingredient a compound represented by the following formula (2):

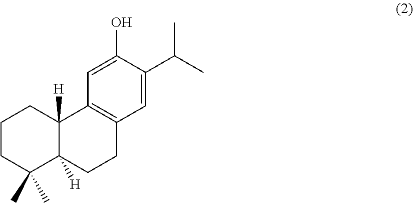

[4] The antibacterial agent according to any one of the above [1] to [3], which has antibacterial activity against gram-positive bacteria.
[5] The antibacterial agent according to the above [4], wherein the gram-positive bacteria are bacteria belonging to any genus selected from the group consisting of the genus Staphylococcus, the genus Lactobacillus, the genus Listeria, the genus Alicyclobacillus and the genus Bacillus.
[6] The antibacterial agent according to the above [4] or [5], wherein the gram-positive bacteria are bacteria belonging to any species selected from the group consisting of Staphylococcus aureus, Lactobacillus plantarum, Listeria monocytogenes, Alicyclobacillus acidoterrestris and Bacillus subtilis.
[7] An antibacterial agent comprising as an active ingredient a compound represented by the following formula (1), used for disinfection against bacteria belonging to the genus Listeria:

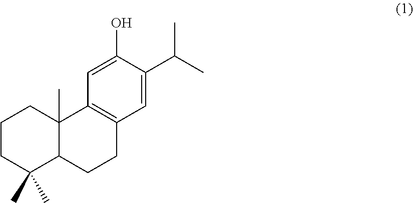

[8] An antibacterial agent comprising at least one of compounds represented by the following formulae (1) and (2) in an amount of at least 50 ppm:

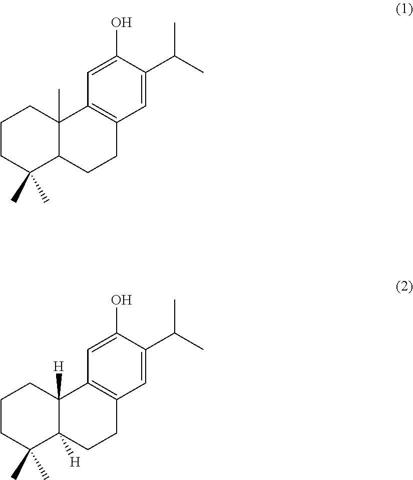

[9] The antibacterial agent according to the above [8], having the compound represented by the formula (1) or (2) dissolved or dispersed in water, an alcohol, edible fat or oil, or a mixture thereof.
[10] The antibacterial agent according to the above [8] or [9], wherein at least one of the compounds represented by the formulae (1) and (2) is extracted from a labiatae plant.
[11] The antibacterial agent according to any one of the above [1] to [10], which further contains a hop extract.
[12] The antibacterial agent according to any one of the above [1] to [11], which is used for disinfection for foods.
[13] A disinfecting method, which comprises incorporating the antibacterial agent as defined in any one of the above [1] to [12] in a sample.

### EFFECTS OF THE INVENTION

By using the antibacterial agent of the present invention, sufficient antibacterial effects are exhibited against gram-positive bacteria such as the genus Staphylococcus, the genus Lactobacillus, the genus Listeria, the genus Alicyclobacillus and the genus Bacillus, particularly bacteria belonging to the genus Listeria, only with a very small addition amount to a sample (object). The antibacterial agent of the present invention is widely useful in the field of foods, beverage, medical and pharmaceutical supplies, and environmental safety, and is particularly useful as a food preservative, and the disinfecting method of the present invention is particularly useful as a method of storing foods.

### BEST MODE FOR CARRYING OUT THE INVENTION

Now, the present invention will be described in detail. However, this description is only examples (representative examples) of the present invention, and the present invention is not limited thereto within the scope of the invention.

The antibacterial agent of the present invention comprises as an active ingredient a specific compound contained in a labiatae plant extract, i.e. either one or both of compounds represented by the above formulae (1) and (2). Both the compounds represented by the formulae (1) and (2) are water-insoluble compounds contained in a labiatae plant extract.

In the present invention, the labiatae plant extract is not limited and may, for example, be at least one extract selected from the group consisting of a rosemary extract, a sage extract, a thyme extract and an oregano extract, and among them, a rosemary extract is particularly preferred.

Rosemary, sage, thyme and oregano are plants belonging to the same order and contain the same components. Further, they are plants belonging to the family Labiatae, and extracts from plants belonging to the same family contain substantially the same components although the proportion of the components may vary.

Further, the portion to be subjected to extraction is not limited and may be at least one portion selected from the group consisting of leaves, stems, flowers and roots, and is properly selected in accordance with the type of the labiatae plants, the extraction method, the extraction conditions, etc. Particularly an extract obtained from leaves or stems is preferred.

The labiatae plant extract in the present invention is not limited so long as it is a labiatae plant extract, and is particularly preferably a water-insoluble extract. It is more preferably a water-insoluble labiatae plant extract having a solubility of less than 0.1 g in 100 g of water at 25° C., preferably a water-insoluble rosemary extract. The solubility in water is preferably at most 0.05 g, more preferably at most 0.01 g in 100 g of water at 25° C.

The labiatae plant extract can be prepared, for example, by the following method, but the method is not limited to the following method.

Firstly, leaves and stems of the above labiatae plant, e.g. rosemary, are dried and subjected to extraction with hexane, ethyl acetate, ethanol, an ether, alkali water, neutral water, acid water or a mixture thereof. Otherwise, extraction is possible with a supercritical or subcritical fluid, for example, such as carbon dioxide in such a state. A preferred extraction method is an extraction method with hexane, hexane/ethanol, ethanol or ethanol/water.

Usually, extraction is carried out using the above solvent in an amount of 10 times per 1 kg of the labiatae plant such as rosemary at from 5 to 80° C., preferably at from 40 to 70° C. with stirring, followed by filtration to obtain a filtrate. Further, the residue is washed with the same solvent several times to obtain a filtrate. These filtrates are mixed, and water in the same amount as the mixed filtrate is added to precipitate a deposit, activated carbon in an amount one tenth the amount of the filtrate is added, the filtrate is stirred for 1 hour and preserved at a cold place overnight, and the deposit is removed by filtration. This operation is repeatedly carried out several times, and the obtained filtrates are mixed and concentrated under reduced pressure to obtain a water-insoluble labiatae plant extract.

Further, extraction without use of a liquid for extraction is possible. For example, a labiatae plant is heated and pressed or squeezed to obtain a labiatae plant extract.

The method for extraction of the compound of the above formula (1) or (2) constituting the antibacterial agent of the present invention is not limited. For example, the above labiatae plant extract is made to flow through an ODS (octadecylsilyl silica gel) column (30 mm in diameter×120 mm) with an eluent CH₃CN, and then components remaining in the resin is advanced by chloroform/methanol=100/0 or 99/1 (volume ratio) to obtain a crude fraction extract when chloroform 100% is made to flow. Further, by washing with chloroform/methanol=99/1 (volume ratio), a crude fraction extract can be obtained. There are two types of components obtainable by this method, one is ferruginol (the compound of the above formula (1), hereinafter this will sometimes be referred to as “Terpenoid-1”), and the other is a component novel as a natural product (the compound of the above formula (2), hereinafter this will sometimes be referred to as “Terpenoid-2”).

These compounds can be identified by NMR (¹H-NMR) (¹H-nuclear magnetic resonance). The NMR data and the mass spectrum data of the compound of the formula (2) as a novel component are as follows.

Terpenoid-2

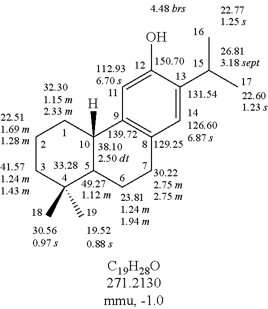

In the present invention, the compounds represented by the above formulae (1) and (2) may be an extract or may be a compound isolated from the extract or a synthetic compound. In other words, the antibacterial agent of the present invention is not limited to an extract from a labiatae plant so long as it contains the compound of the above formula (1) or (2). In a case of using an extract, the extract may be used as an antibacterial agent as it is, but the extract is preferably concentrated, isolated and purified. Particularly, it is possible to increase the concentration of the compound of the above formula (1) or (2) by extracting the compound of the above formula (1) or (2) from the labiatae plant extract, followed by at least one step selected from the group consisting of concentration, extraction, precipitation, isolation and purification to obtain the compound of the above formula (1) or (2), whereby the antibacterial properties aimed in the present invention can more remarkably be exhibited.

These two compounds have antibacterial activity against gram-positive bacteria particularly bacteria belonging to the genus Listeria, and can be used as an active ingredient of an antibacterial agent.

From labiatae plants, various terpenoids have already been isolated and identified. Among them, carnosic acid is known to have an anti-listeria effect. However, that the above Terpenoid-1 and Terpenoid-2 are an active ingredient for antibacterial activity of a water-insoluble labiatae plant extract and that these compounds have excellent antibacterial activity particularly against bacteria belonging to the genus Listeria, are first found in the present invention as far as the present inventors are aware. Further, Terpenoid-2 is a novel substance as a natural product, and its antibacterial effect is also first found in the present invention.

The antibacterial agent of the present invention may be used in any formulation such as a solution, a dispersion, a powder or granules. Accordingly, it can be used as a composition containing the compound represented by the above formula (1) or (2). In a case where the antibacterial agent of the present invention is in the form of a solution or a dispersion, considering convenience at the time of use, a solution or dispersion having the compound represented by the above formula (1) or (2) dissolved or dispersed in water, an alcohol such as ethanol, edible fat or oil, or a mixture thereof may preferably be used.

In a case where the antibacterial agent of the present invention is in the form of a composition, the content of the compound represented by the above formula (1) or (2) is not limited, but the composition preferably contains at least either one of these compounds in an amount of usually at least 50 ppm, preferably at least 100 ppm, more preferably at least 200 ppm. If the content of the compound in the composition is less than the above lower limit, no sufficient effect as an antibacterial agent may sometimes be obtained. The upper limit of the content of the compound in the composition is not limited, however, since the antibacterial agent of the present invention has a sufficient effect only with a very slight amount, a sufficient antibacterial effect can be obtained even with a content of usually at most 5 wt %, further at most 2 wt %, particularly at most 1 wt %.

Further, in a case where the compounds of the above formulae (1) and (2) are used in combination, the total content of these compounds in the composition is also not limited, and it is usually at least 50 ppm, preferably at least 100 ppm, more preferably at least 200 ppm. In a case where the total content of these compounds in the composition is less than the above lower limit, no sufficient effect as an antibacterial agent may sometimes be obtained. The upper limit of the total content of these compounds in the composition is not limited, however, since the antibacterial agent of the present invention has a sufficient effect even with a very small amount, a sufficient antibacterial effect can be obtained even with a content of usually at most 5 wt %, further at most 2 wt %, particularly at most 1 wt %. In a case where the total content of these compounds in the composition exceeds the above upper limit, there may be a case where it is an amount sufficient to obtain a required antibacterial effect.

As described above, as labiatae plant extracts, known substances such as terpenoids have already been isolated and identified. These components can be obtained by adjusting the extraction solvent, the extraction conditions, etc. in the same manner as the above extraction and fractionating method to obtain Terpenoid-1 and Terpenoid-2. As components contained in the labiatae plant extract, as a water-insoluble extract, 7α-ethoxyrosmanol, carnosol, carnosic acid (12-methoxy-trans-carnosic acid), rosmanol, epirosmanol, betulin or betulinic acid may, for example, be mentioned. One or more of these components may be combined and incorporated in the composition in combination. When at least one component selected from the group consisting of rosmanol, carnosil, carnosic acid and betulinic acid is used as a component used in combination with Terpenoid-1 and/or Terpenoid-2, the antibacterial effect of the antibacterial agent of the present invention may sometimes be enhanced. Further, a water-soluble labiatae plant extract may be used in combination in the composition, and for example, rosmalinic acid may, for example, be mentioned.

Further, for the antibacterial agent of the present invention, as the case requires, an emulsifier, an extender or the like may be added.

The emulsifier may, for example, be a polyglyceryl fatty acid ester such as polyglyceryl laurate, polyglyceryl myristate, polyglyceryl palmitate, polyglyceryl stearate, polyglyceryl oleate or polyglyceryl behenate; a sucrose fatty acid ester such as sucrose octanoate, sucrose decanoate, sucrose laurate, sucrose myristate, sucrose palmitate, sucrose stearate or sucrose oleate; a polysorbate such as polysorbate 40, 60, 65 or 80; or lecithine degradation product, and two or more of them may be used in combination. The content of the emulsifier is not limited, and is usually from 0.001 to 1,000 wt %, preferably from 0.01 to 90 wt %, more preferably from 0.1 to 50 wt %, further preferably from 1 to 20 wt % to the total amount of Terpenoid-1 and Terpenoid-2.

The extender may, for example, be dextrin, α-cyclodextrin, β-cyclodextrin, γ-cyclodextrin, modified starch or a saccharide such as sucrose or lactose; a sugar alcohol such as oligotose, trehalose, xylitol or erythritol; or a triterpene such as betulinic acid or a betulinic acid ester derivative, and two or more of them may be used in combination. The content of the extender is not particularly limited and is usually from 0.01 to 1,000 wt %, preferably from 0.1 to 90 wt %, more preferably from 0.5 to 50 wt %, further preferably from 1 to 20 wt % to the total amount of Terpenoid-1 and Terpenoid-2.

In the present invention, another antioxidant such as vitamin C, tocopherol, vitamin P, chlorogenic acid, coffee bean extract, sunflower extract, grape seed extract, α-G rutin, catechin, green tea extract or rosemary extract may be used in combination. Two or more of them may be used in combination, and the content is not limited, and is usually from 0.01 to 1,000 wt %, preferably from 0.1 to 50 wt %, more preferably from 0.5 to 30 wt %, further preferably from 1 to 20 wt %, to the total amount of Terpenoid-1 and Terpenoid-2.

The antibacterial agent of the present invention has effective antibacterial activity against microorganisms, and is particularly preferably used against gram-positive bacteria such as bacterias belonging to any genus selected from the genus Staphylococcus, the genus Lactobacillus, the genus Listeria, the genus Alicyclobacillus and the genus Bacillus.

Particularly, the antibacterial agent of the present invention has a remarkable effect against bacteria belonging to the species selected from the group consisting of Staphylococcus aureus, Lactobacillus plantarum, Listeria monocytogenes, Alicyclobacillus acidoterrestris and Bacillus subtilis.

Further, the antibacterial agent comprising the compound represented by the formula (1) or (2) as an active ingredient is particularly effective as an antibacterial agent against bacterial belonging to the genus Listeria, such as Listeria monocytogenes.

The antibacterial agent of the present invention can be widely used in the fields of foods, beverage, medical and pharmaceutical supplies, and environmental safety, and is particularly useful as a food preservative. By adding the antibacterial agent of the present invention to various samples in such fields, antibacterial activity can be exhibited in the samples. In the present invention, a sample means an object to which the antibacterial agent of the present invention is applied.

As described above, an antibacterial agent extracted from natural products, having high antibacterial activity against bacteria belonging to the genus Listeria, and free from an unpleasant odor, has not been confirmed except for the antibacterial agent of the present invention.

As a sample to which the antibacterial agent of the present invention is added, foods in which microorganisms will easily grow are effective, and their specific examples include fish, meat and fat and oil processed foods. Particularly, the antibacterial agent of the present invention can be preferably used for fish, meat, and fat and oil processed foods, which are likely to be deteriorated, and fish, meat, and fat and oil processed foods which are stored for a long period of time. Their specific examples include fresh fish, dried fish, overnight dried fish, dried seasoned fish, shellfish, red snapper, crustacean, minced fish, fish paste products, delicacies, fish sausages, salt cured products, dried layer, seaweed foods, chicken, pork, beef, mutton, sausages, ham and their processed products. Further, the antibacterial agent of the present invention can be applied not only for human foods but also pet foods and foodstuff. Further, it can be used as an antibacterial agent not only for meat and fish but also for prevention of decay of agricultural products (such as vegetables, root vegetables and fruits) produced utilizing the soil.

The concentration of the antibacterial agent of the present invention added to a sample is usually from 0.01 to 10,000 ppm, preferably from 0.1 to 5,000 ppm, more preferably from 1 to 1,000 ppm as the total amount of either one or both of the compounds represented by the above formulae (1) and (2).

Further, the antibacterial agent of the present invention may be used in combination with other antibiotics such as carnosic acid already known as a labiatae plant extract, hop acid which is a hop extract, niacin (niacin A, niacin Z), polylysine, milt protein or lysozyme, thereby to obtain an antibacterial agent having more excellent antibacterial activity.

Particularly a combined use of the antibacterial agent of the present invention with a hop extract such as hop acid is useful as an antibacterial agent derived from natural products, whereby the specific odor of hop acid is reduced, and high antibacterial property will be achieved.

The antibacterial agent of the present invention is useful not only for disinfection of foods but also as a microbicide used in e.g. a food handling environment. Specifically, it can be used as a microbicide to keep hands and clothes of food handlers, cooking devices, the kitchen, etc. clean. In the case of such an application, it is preferably used in the form of a powder, a liquid, a spray or the like.

### EXAMPLES

Now, the present invention will be described in further detail with reference to Examples. However, it should be understood that the present invention is by no means restricted to such specific Examples.

### Experimental Example 1

### Preparation of Water-Insoluble Rosemary Extract

To 1 kg of rosemary, 10 L of 50 wt % hydrous ethanol was added, followed by heat reflux for 3 hours, and the liquid was subjected to filtration in the warm conditions to obtain a filtrate. The residue was further subjected to extraction treatment twice with 6 L of 50 wt % hydrous ethanol in the same manner to obtain a filtrate. These obtained filtrates were mixed, and 5 L of water was added to precipitate a deposit. 100 g of activated carbon was added thereto, and the mixture was stirred for 1 hour, stored at a cold place overnight and subjected to filtration to obtain a mixture of the deposit and the activated carbon. 4 L of ethanol was added to the mixture, followed by heat reflux for 3 hours, and the liquid was subjected to filtration in the warm conditions to obtain a filtrate. The residue was further subjected to extraction treatment twice with 2.4 L of ethanol in the same manner to obtain a filtrate. These filtrates were mixed and concentrated under reduced pressure to distill ethanol off thereby to obtain a powdery water-insoluble rosemary extract (content of carnosol and carnosic acid: 24.9 wt %).

### (Isolation and Identification of Active Ingredient of Water-Insoluble Rosemary Extract)

The water-insoluble rosemary extract was made to flow through an ODS (octadecylsilyl silica gel) column (30 mm in diameter×120 mm) with acetonitrile as an eluent, and components remaining in the resin was advanced with chloroform/methanol (volume ratio)=100/0, 99/1 to obtain a crude fraction rosemary extract when chloroform 100% was made to flow. Further, after washing with chloroform/methanol (volume ratio)=99/1, two types of components were obtained. These components were subjected to structural analysis by ¹H-NMR and as a result, it was found that one is ferruginol (Terpenoid-1, compound of the formula (1)) and the other is a component novel as a natural product (Terpenoid-2, compound of the formula (2)).

The NMR (¹H-NMR) data and the mass spectrum data of Terpenoid-2 as a novel component are as follows.

Terpenoid-2

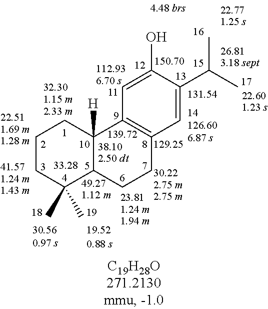

(Antibacterial Activity Against Listeria monocytogenes)

The components (Terpenoid-1 and Terpenoid-2) isolated from the water-insoluble rosemary extract were dissolved in DMSO (dimethylsulfoxide) at a concentration of 2,000 ppm to obtain samples for antibacterial activity test.

The antibacterial test was carried out as follows using Listeria monocytogenes JCM2873.

### (1) Preparation of Culture Fluid

8.95 ml of Mueller Hinton Broth (MHB) culture medium was put in a test tube and sterilized in an autoclave. The culture medium concentration was adjusted to achieve a specified concentration when 50 μl of the sample was added.

### (2) Preparation of Bacteria Liquid

Listeria monocytogenes JCM2873 was cultured by shake culture at 37° C. for 1 day in the MHB culture medium, and diluted to an appropriate bacteria concentration (3.1×10⁴ cfu/ml) by the MHB culture medium.

### (3) Antibacterial Test

To the test tube in which 8.95 ml of the MHB culture medium prepared in the above (1) was put, 50 μl of the sample and 1 ml of the bacteria liquid prepared in the above (2) were added and stirred (isolated component concentration at the time of test: 10 ppm) and left at rest at 37° C. for 24 hours. As a negative control and a positive control, sterilized water and erythromycin (solution at a concentration of 2,000 ppm in ethanol) were added instead of the sample.

### (4) Judgment

From the test tube left at rest at 37° C. for 24 hours, a loopful of the culture fluid was collected and smeared on a Mueller Hinton agar medium. After culturing at 37° C. for 2 days, formation of colonies on the agar medium and its degree were judged.

As a result, on the negative control culture medium to which sterilized water was added, a large number of colonies (3×10⁸ cfu/ml) were formed. On the other hand, on the culture medium to which samples (Terpenoid-1 and/or Terpenoid-2) were added and on the positive control culture medium, no formation of colonies was confirmed. From these results, Terpenoid-1 and Terpenoid-2 were found to have excellent antibacterial activity.

### Experimental Example 2

### Fractionation of Extract Components

In the same manner as in Experimental Example 1, 902 mg of a powdery water-insoluble rosemary extract was obtained, which was dissolved in a small amount of acetonitrile, and the solution was injected into a column (30 mm in diameter×120 mm) filled with 30 g of ODS (octadecylsilyl silica gel), and then eluted by a step gradient method using water/acetonitrile mixed liquids. The mixing ratios of the eluents were such that water/acetonitrile (volume ratio)=80:20, 60:40, 40:60, 20:80 and 0:100, in an amount of 150 ml each. Then, 150 ml of chloroform was made to flow through the column, and further 150 ml of methanol was made to flow.

The eluates with water/acetonitrile (volume ratio)=60:40, 40:60 and 20:80 were subjected to fractionation with silica gel thin layer chromatography (TLC), and the respective components were analyzed by NMR (¹H-NMR) and identified as shown in Table 1.

All the eluates with acetonitrile (100%), chloroform and methanol were mixed and concentrated to dryness, and further subjected to the following fractionation by silica gel. That is, the dried residue was dissolved in chloroform and injected into a column filled with 15 g of a silica gel, and eluated by a step gradient method with chloroform/methanol mixed liquids. The mixing ratios of the eluates were such that chloroform/methanol (volume ratio)=100:0, 99:1, 98:2, 97:3, 96:4, 95:5 and 0:100, in an amount of 150 ml each.

Each eluate was concentrated to dryness and further subjected to fractionation by high performance liquid chromatography (HPLC). For HPLC, an ODS column was used, and a water/methanol mixed solution (volume ratio 10:90) was used. Dried residues of the eluates with chloroform/methanol (volume ratio)=98:2, 96:4 and 0:100 were analyzed by NMR (¹H-NMR) and identified as shown in Table 1.

TABLE 1

| | | | | | Amount |
|---|---|---|---|---|---|
| | Composition of eluate | | | | collected |
| Column | (volume ratio) | | Fraction No. | Fraction component | (mg) |
| ODS | Water 60 | Acetonitrile 40 | 1 | Rosmanol | 3.6 |
| | | | | Epirosmanol | 1.6 |
| | Water 40 | Acetonitrile 60 | 2 | 7α-ethoxyrosmanol | 5.6 |
| | | | | Carnosol | 14.5 |
| | Water 20 | Acetonitrile 80 | 3 | Carnosol | 2.7 |
| | | | | 12-methoxy-trans- | 3.0 |
| | | | | carnosic acid | |
| Silica | Chloroform 98 | Methanol 2 | 4 | Betulinic acid | 5.2 |
| gel | Chloroform 96 | Methanol 4 | 5 | Betuline | 4.4 |
| | Chloroform 0 | Methanol 100 | 6 | Terpenoid-1 | 4.8 |
| | | | | Terpenoid-2 | 2.3 |

(Antibacterial Activity Against Listeria monocytogenes)

The above obtained isolated components were diluted with dimethylsulfoxide (manufactured by KANTO CHEMICAL CO., INC.) to prepare three test liquids at fraction concentrations (wt %) of 2%, 1% and 0.5%. As positive controls, erythromycin (manufactured by Wako Pure Chemical Industries Ltd.) was dissolved in ethanol (Junsei Chemical Co., Ltd.) to have a concentration of 1,000 ppm, 500 ppm or 250 ppm. As a negative control, sterilized water was used.

The antibacterial test was carried out by using Listeria monocytogenes ATCC49594 as follows.

(1) 8.9 ml of a brain heart infusion (BHI) culture medium (manufactured by NISSUI PHARMACEUTICAL CO., LTD.) was put in a test tube and treated in an autoclave (manufactured by TOMY SEIKO CO., LTD., TOMY BS-325). The culture medium concentration was adjusted to achieve a specified concentration when 0.1 ml of the above test liquid was added.

### (2) Preparation of Bacteria Liquid

Listeria monocytogenes ATCC 49594 was cultured in the BHI culture medium by shake culture at 37° C. for 1 hour and diluted to an appropriate bacteria concentration (2.0×10³ cfu/ml) by the BHI culture medium.

### (3) Antibacterial Test

To the test tube in which 8.9 ml of the BHI culture medium prepared in the above (1) was put, 0.1 ml of the prepared test liquid and 1 ml of the bacteria liquid prepared in the above (2) were added, stirred and left at rest at 30° C. for 24 hours.

### (4) Evaluation of Inhibitory Ratio

The turbidity of the culture fluid in the test tube left at rest at 30° C. for 24 hours was measured by a turbidimeter (manufactured by TAITEC CORPORATION, miniphoto 518R) at a wavelength of 660 nm. The inhibitory ratio was calculated from the following formula, where A is the turbidly in the case of a culture fluid to which 0.1 ml of dimethylsulfoxide was added instead of the test liquid.

Inhibitory ratio (%)=[(A−[turbidity of culture fluid])/A]×100

### (5) Evaluation of Bacteria Growth

The culture fluid in the test tube left at rest at 30° C. for 24 hours was diluted with a phosphate buffer solution (PBS) and smeared on a BHI agar medium. After culturing at 37° C. for 24 hours, the number of bacteria on the agar medium was measured by observation with a microscope. With respect to the dilution with PBS, the culture fluid was preliminarily properly diluted within a range of from 10² to 10⁹ times to such a concentration that the number of bacteria can be counted with a microscope 24 hour later (the number of bacteria 24 hours later was calibrated in accordance with the proportion preliminarily diluted with PBS).

The results of evaluation of the inhibitory ratio and the bacteria growth evaluation of the culture fluids containing erythromycin, Terpenoid-1 and Terpenoid-2 are shown in Table 2. Erythromycin which is a synthetic antibacterial agent used as the positive control exhibited high antibacterial property within a concentration range of from 2.5 to 10 ppm. Terpenoid-1 and Terpenoid-2 from a natural extract are also confirmed to have antibacterial activity against Listeria monocytogenes at a concentration of 50 ppm or higher. Further, a remarkable antibacterial activity was exhibited by increasing the concentration of Terpenoid-1 or Terpenoid-2.

The results of the inhibitory ratio and the bacteria growth evaluated under the same conditions as for Terpenoid-1 and Terpenoid-2, with respect to extracted components other than Terpenoid-1 and Terpenoid-2, are shown in Table 3. As a result, all of 7α-ethoxyrosmanol, carnosol, 12-methoxy-tarns-carnosic acid, rosmanol, epirosmanol and betulinic acid had low or no antibacterial activity against Listeria monocytogenes.

TABLE 2

| | | Concentration of | | | |
|---|---|---|---|---|---|
| | | added substance | Turbidity of | Inhibitory | Number of |
| | | (ppm) | culture fluid | ratio (%) | bacteria (cfu/ml) |
| Water | | — | 0.566 | — | 5.9 × 10¹⁰ |
| Dimethylsulfoxide | | — | 0.509 | Standard | 6.8 × 10⁹ |
| Added | Erythromycin | 2.5 | 0.011 | 97.8 | 3.7 × 10² |
| substance | | 5 | 0.022 | 95.6 | 9.5 × 10² |
| | | 10 | −0.008 | 98.4 | 1.8 × 10² |
| | Terpenoid-1 | 50 | 0.485 | 4.6 | 3.8 × 10⁹ |
| | | 100 | 0.290 | 43.0 | 1.6 × 10⁹ |
| | | 200 | −0.003 | 100 | 3.8 × 10⁶ |
| | Terpenoid-2 | 50 | 0.472 | 7.2 | 4.4 × 10⁹ |
| | | 100 | 0.402 | 21.0 | 2.5 × 10⁹ |
| | | 200 | 0.268 | 47.2 | 1.3 × 10⁹ |

TABLE 3

| | | Concentration of | | Number of |
|---|---|---|---|---|
| | | added substance | Inhibitory | bacteria |
| | | (ppm) | ratio (%) | (cfu/ml) |
| Added | 7α- | 50 | 4.0 | 2.5 × 10⁹ |
| substance | Ethoxyrosmanol | 100 | 0.5 | 3.6 × 10⁹ |
| | | 200 | 2.6 | 3.8 × 10⁹ |
| | Carnosol | 50 | 0.0 | 4.3 × 10⁹ |
| | | 100 | 0.0 | 4.7 × 10⁹ |
| | | 200 | 0.0 | 3.9 × 10⁹ |
| | 12-Methoxy- | 50 | 11.8 | 3.1 × 10⁹ |
| | tarns-carnosic | 100 | 17.7 | 4.1 × 10⁹ |
| | acid | 200 | 20.6 | 4.3 × 10⁹ |
| | Rosmanol | 50 | 10.2 | 3.9 × 10⁹ |
| | | 100 | 5.1 | 3.7 × 10⁹ |
| | | 200 | 16.0 | 3.9 × 10⁹ |
| | Epirosmanol | 50 | 0.0 | 4.0 × 10⁹ |
| | | 100 | 0.0 | 4.2 × 10⁹ |
| | | 200 | 0.0 | 3.2 × 10⁹ |
| | Betulinic acid | 50 | 0.0 | 4.0 × 10⁹ |
| | | 100 | 0.0 | 4.5 × 10⁹ |
| | | 200 | 0.0 | 3.0 × 10⁹ |

### INDUSTRIAL APPLICABILITY

The antibacterial agent of the present invention can be obtained from natural products, has high antibacterial activity particularly against bacteria belonging to the genus Listeria with a small content, and is free from an unpleasant odor. Thus, it is useful as an antibacterial substance in wide fields including the fields of foods, beverage, medical and pharmaceutical supplies, and environmental safety.

Considering the energy reduction, the global environment planning, the population increase planning, the food problem, etc. in recent years, by using the antibacterial agent of the present invention, particularly decay of fresh foods and beverage can be suppressed, and it is possible to effectively utilize food and beverage. That is, the antibacterial agent of the present invention is useful in various industrial fields such as livestock industry, marine products industry, food processing, food and beverage manufacturing, food and beverage selling, distribution, medical care and sanitation.

The entire disclosure of Japanese Patent Application No. 2008-201147 filed on Aug. 4, 2008 including specification, claims and summary is incorporated herein by reference in its entirety.

1. An antibacterial agent comprising as an active ingredient either one or both of compounds represented by the following formulae (1) and (2) contained in a labiatae plant extract:

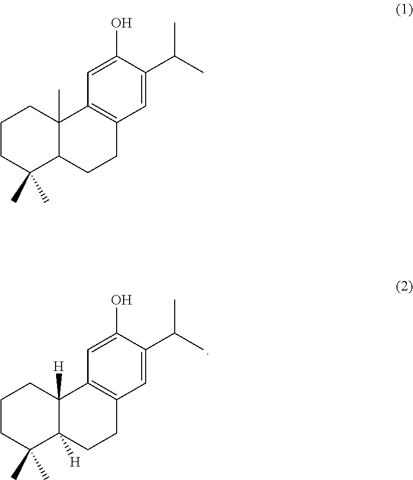

2. The antibacterial agent according to claim 1, wherein the labiatae plant extract is at least one extract selected from the group consisting of a rosemary extract, a sage extract, a thyme extract and an oregano extract.

3. An antibacterial agent comprising as an active ingredient a compound represented by the following formula (2):

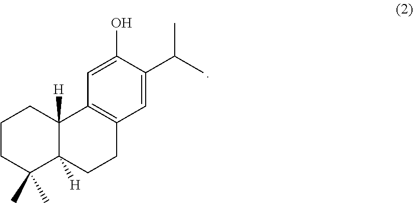

4. The antibacterial agent according to claim 1, which has antibacterial activity against gram-positive bacteria.

5. The antibacterial agent according to claim 4, wherein the gram-positive bacteria are bacteria belonging to any genus selected from the group consisting of the genus Staphylococcus, the genus Lactobacillus, the genus Listeria, the genus Alicyclobacillus and the genus Bacillus.

6. The antibacterial agent according to claim 4, wherein the gram-positive bacteria are bacteria belonging to any species selected from the group consisting of Staphylococcus aureus, Lactobacillus plantarum, Listeria monocytogenes, Alicyclobacillus acidoterrestris and Bacillus subtilis.

7. An antibacterial agent comprising as an active ingredient a compound represented by the following formula (1), used for disinfection against bacteria belonging to the genus Listeria:

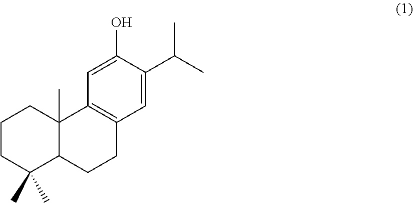

8. An antibacterial agent comprising at least one of compounds represented by the following formulae (1) and (2) in an amount of at least 50 ppm:

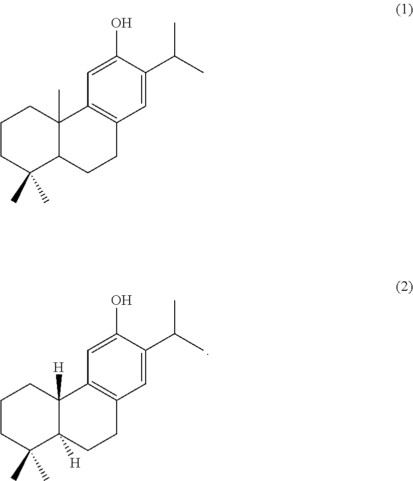

9. The antibacterial agent according to claim 8, having the one compound represented by the formula (1) or (2) dissolved or dispersed in water, an alcohol, edible fat or oil, or a mixture thereof.

10. The antibacterial agent according to claim 8, wherein at least one of the compounds represented by the formulae (1) and (2) is extracted from a labiatae plant.

11. The antibacterial agent according to claim 1, which further contains a hop extract.

12. The antibacterial agent according to claim 1, which is used for disinfection for foods.

13. A disinfecting method, which comprises incorporating the antibacterial agent as defined in claim 1 in a sample.

14. The antibacterial agent according to claim 7, which further contains a hop extract.

15. The antibacterial agent according to claim 8, which further contains a hop extract.

16. The antibacterial agent according to claim 7, which is used for disinfection for foods.

17. The antibacterial agent according to claim 8, which is used for disinfection for foods.

18. A disinfecting method, which comprises incorporating the antibacterial agent as defined in claim 7 in a sample.

19. A disinfecting method, which comprises incorporating the antibacterial agent as defined in claim 8 in a sample.
